(19) United States
(12) Patent Application Publication
(10) Pub. No.: US 2011/0092465 A1
(43) Pub. Date: Apr. 21, 2011

(54) MONOMERIC PYROPHOSPHATE COMPLEXES AND METHODS OF TREATMENT USING THE COMPLEXES

(75) Inventor: Robert Doyle, Syracuse, NY (US)

(73) Assignee: SYRACUSE UNIVERSITY, Syracuse, NY (US)

(21) Appl. No.: 12/909,188

(22) Filed: Oct. 21, 2010

Related U.S. Application Data
(60) Provisional application No. 61/253,815, filed on Oct. 21, 2009.

(51) Int. Cl. A61K 31/66; C07F 9/09; A61P 35/00

(52) U.S. Cl. 514/106; 556/17

(57) ABSTRACT

The present invention involves pyrophosphate bridged coordination complexes and the treatment of medical conditions, such as cancer, using the pyrophosphate bridged coordination complexes. The pyrophosphate bridged coordination complexes include four new compounds, [Co(phen)₂(H₂P₂O₇)].4H₂O (1.4H₂O), [Ni(phen)₂(H₂P₂O₇)].8H₂O (2.8H₂O), [Cu(phen)(H₂O)(H₂P₂O₇)], and {[Cn(phen)(H₂O)(P₂O₇)] [Na₂(H₂O)₈]}.6H₂O(4.14H₂O) found effective for treating cancer cells. The pyrophosphate bridged coordination complexes also include three previously reported compounds, {[Ni(phen)₂]₂(μ-P₂O₇)}.27H₂O (compound 11), {[Cu(phen)(H₂O)]₂(μ-P₂O₇)}.8H₂O (compound 12), and {[Co(phen)₂]₂(μ-25 P₂O₇)}.6MeOH (compound 13), (where phen is 1,1 0′-phenanthroline), whose effectiveness in treating cancer cells was previously unknown.

### CROSS-REFERENCE TO RELATED APPLICATIONS

The present application claims priority to U.S. Provisional App. No. 61/253,815, filed on Oct. 21, 2009.

### BACKGROUND OF THE INVENTION

1. Field of the Invention

The present invention relates to pyrophosphate complexes and, more particularly, to four new monomeric phosphate complexes and three known pyrophosphate coordination complexes that may be used to treat diseases.

2. Description of the Related Art

Metals have enormous potential for application in medicine, offering real possibilities for new therapies with diverse mechanisms of action. The field of metallopharmaceuticals has already demonstrated success with gold complexes for the treatment of arthritis, bismuth as an antiulcer agent, and platinum as an antineoplastic agent for example. The latter in particular has paved the way, fueled by cisplatin. Cisplatin, cis-[Pt(NH₃)₂Cl₂], and its second-generation analogues, carboplatin and oxaliplatin, are among the most widely used chemotherapeutic agents worldwide, as one in two cancer patients receives one of these therapeutics. While these drugs have been successful, there are some significant restrictions for their general use. These restrictions include toxicity (including nephrotoxicity, neurotoxicity and associated emetogenesis), and acquired resistance. As a result, the there is a need in the field to expand beyond platinum to include other metals for the treatment and/or earlier diagnosis of cancer.

### BRIEF SUMMARY OF THE INVENTION

It is therefore a principal object and advantage of the present invention to provide metal complexes are useful for treating medical conditions such as cancer.

In accordance with the foregoing objects and advantages, the present invention involves for four new monomeric pyrophosphate complexes, namely [Co(phen)₂(H₂P₂O₇)].4H₂O (1.4H₂O), [Ni(phen)₂(H₂P₂O₇)].8H₂O (2.8H₂O), [Cu(phen)(H₂O)(H₂P₂O₇)], and {[Cu(phen)(H₂O)(P₂O₇)] [Na₂(H₂O)₈]}.6H₂O (4.14H₂O) that show toxicity in certain cancer cell lines. The present invention also involves three pyrophosphate bridged complexes, namely {[Ni(phen)₂]₂(μ-P₂O₇)}.27H₂O, {[Cu(phen)(H₂O)]₂(μ-P₂O₇)}.8H₂O, and {[Co(phen)₂]₂(μ-25 P₂O7)}.6MeOH, (where phen is 1,1 0′-phenanthroline) that also show toxicity in certain cancer cell lines. The present invention thus encompasses the use of pyrophosphate (P2O74-), a polyphosphate anion, as a ligand system for delivering metal compounds for the treatment of medical conditions.

### BRIEF DESCRIPTION OF THE SEVERAL VIEWS OF THE DRAWING(S)

The present invention will be more fully understood and appreciated by reading the following Detailed Description in conjunction with the accompanying drawings, in which:

FIGS. 1A and B are schematics of (A) the 7E-7E stacking interactions between the phen ligands in compound 1, which lead to the formation of 1D motifs running along the crystallographic a axis and linked together in the be plane through hydrogen bonds involving the H₂P₂O₇ groups, and (B) the crystal packing of compound 1 along a;

FIGS. 2A and B are schematics of (A) the 7E-7E stacking interactions between the phen ligands in compound 2 and the face-to-face and edge-to-face along b, OFF face-to-face in the ac plane (zig-zag motif), and (B) a view of the crystal packing of compound 2 along c, showing the hydrogen bond interactions involving the H₂P₂O₇ groups and the water molecules of crystallization, with the solvent trapped into supramolecular channels running along the c axis;

FIGS. 3A and B are schematics of the crystal packing of: (A) compound 3, and (B) compound 4 along the respective crystallographic a axis, where hydrogen atoms on the phen ligands have been omitted for clarity;

FIGS. 4A and B are schematics of (A) the ORTEP plot of the anionic [Cu(phen)(H₂O)(P₂O₇)]⁻² unit in compound 4, and (B) the 1D arrangement of the three crystallographic unique sodium cation in compound 4 (occupancy factor of 0.5 for Na(1) and Na(3)], where the symmetry operation used to generate equivalents is: (A) 1−x, 1−y, 1−z; (B) x, −1+y, z; (C) 1−x, −y, 1−z;

FIGS. 5A through 5C are schematics of (A) the ORTEP plot of compound 5 showing the atom labelling scheme (water molecules of crystallization omitted for clarity), where the thermal ellipsoids are drawn at the 30% probability level, (B) the crystal packing of compound 5 along the b axis (the hydrogen atoms on the phen ligand and on the water molecule of crystallization have been omitted for clarity), and (C) the three dimensional array of hydrogen bonded pyrophosphate ligands and crystallization water molecule in compound 5 (cobalt ions and phenantroline ligands have been omitted for clarity);

FIGS. 6A through 6C are schematics of (A) the ORTEP plot (30% of probability level) of the cation [Ni(phen)₃]²⁺ in the structure of compound 6, (B) the 2D water molecule-chloride ions motif growing in the ac plane of compound 6, and (C) the crystal packing of compound 6 viewed along the crystallographic c axis, where the compound is isostructural with the literature bromide analogous of formula [Ni(phen)₃]Cl₂.8H₂O, differing only in the crystallization water content and hydrogen atoms on the phen ligands and disordered water molecules of crystallization (up or down the water-chloride layers) have been omitted for clarity;

FIG. 7 is a graph of the thermogravimetric analysis compound 1;

FIG. 8 is a graph of the thermogravimetric analysis compound 2;

FIG. 9 is a graph of the thermogravimetric analysis compound 4; and

FIG. 10 is a graph of the thermogravimetric analysis compound 5.

### DETAILED DESCRIPTION OF THE INVENTION

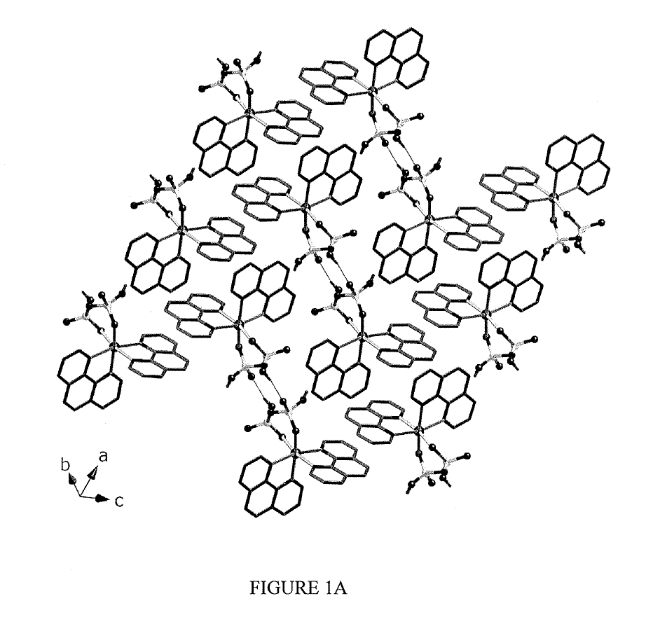

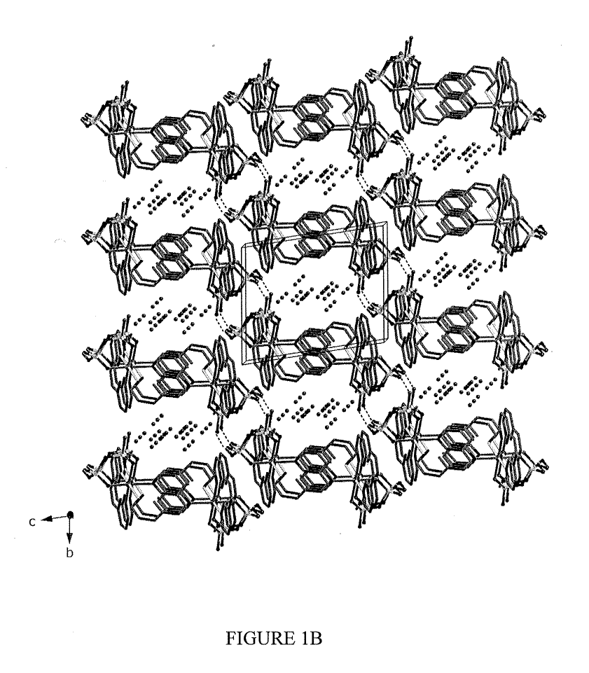

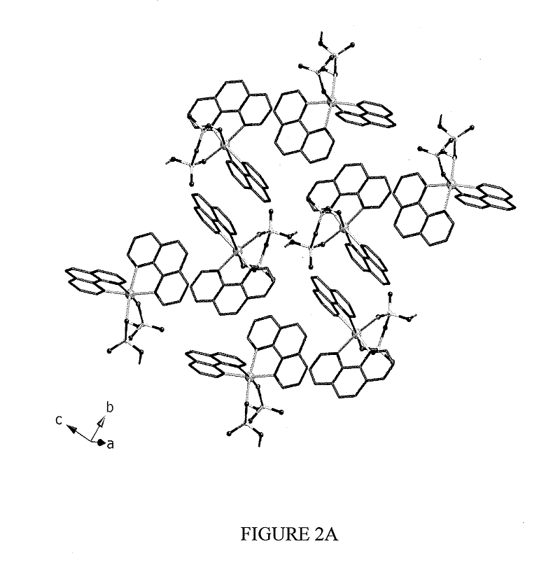

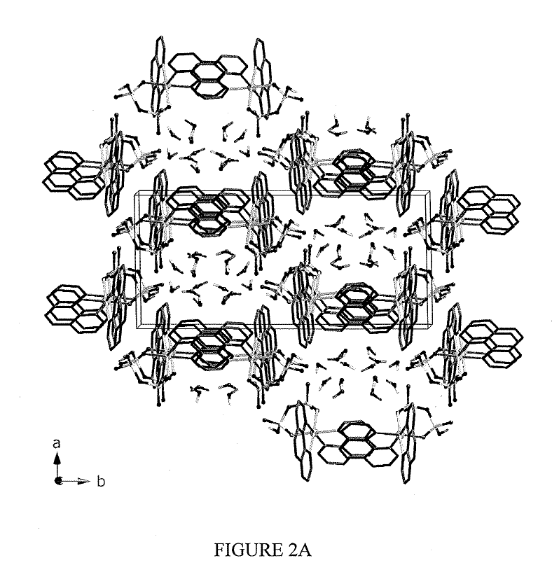

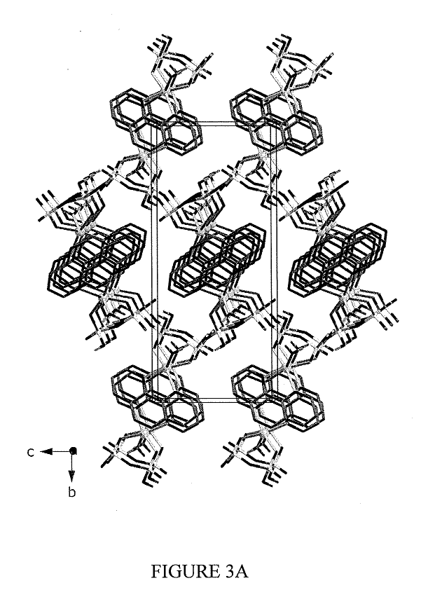

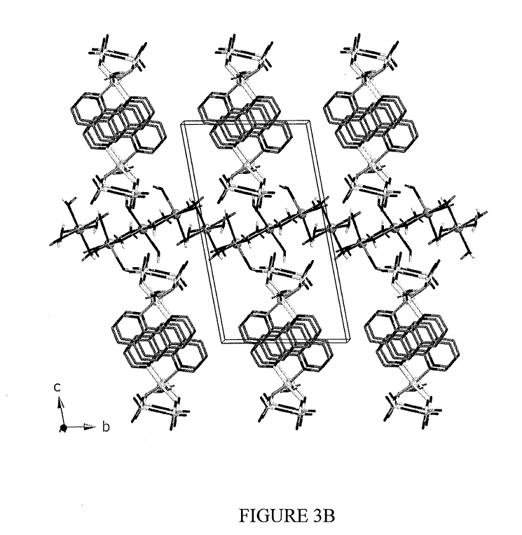

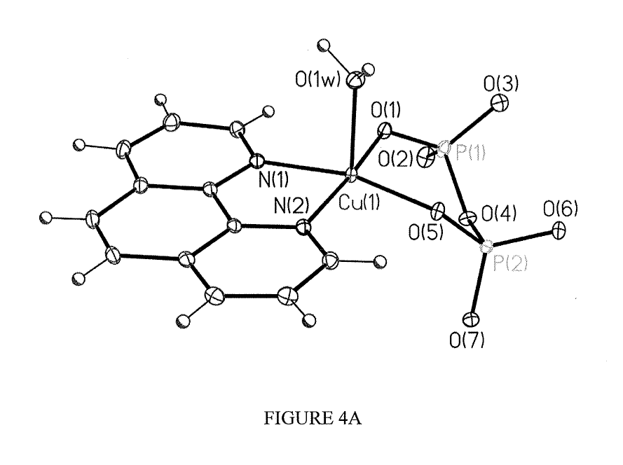

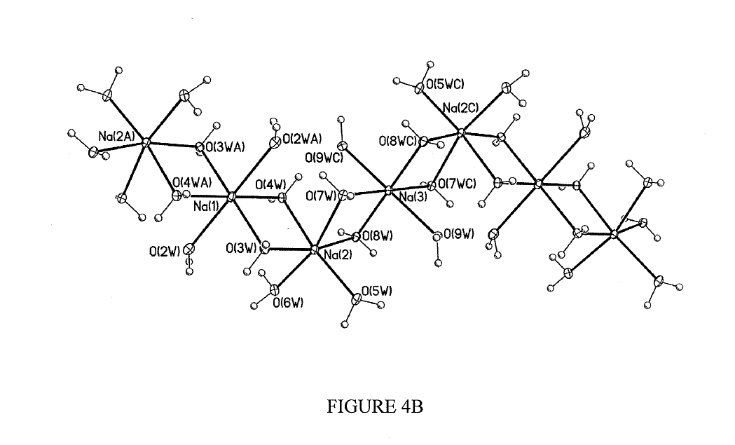

Referring now to the drawings, wherein like reference numerals refer to like parts throughout, the present invention is directed to several new monomeric pyrophosphate complexes. In particular, compound 1, [Co(phen)₂(H₂P₂O₇)].4H₂O (1.4H₂O), is seen in FIGS. 1A and 1B, compound 2, [Ni(phen)₂(H₂P₂O₇)].8H₂O (2.8H₂O) is seen in FIGS. 2A and 2B, compound 3, [Cu(phen)(H₂O)(H₂P₂O₇)], is seen in FIGS. 3A and 3B, and compound 4 {[Cu(phen)(H₂O)(P₂O₇)] [Na₂(H₂O)₈]}.6H₂O (4.14H₂O) is seen in FIGS. 4A and 4B. Compounds 1-3 are all neutral species, with the metal ion coordinated to a dianionic di-hydrogen pyrophosphate group, phen and, in the case of compound 3, a water molecule. Compound 4 is a molecular salt being the dianionic copper(II) complex charge compensated by the presence of two equivalents of sodium cation.

Compounds 1-3 were synthesized from acidic aqueous solutions of CoSO₄.7H₂O, NiCl₂.6H₂O or CuNO₃.2.5H₂O, phen and sodium pyrophosphate typically added in a non-stoichiometric ratio, the pH being lowered with concentrated H₂SO₄ (compound 1), HCl (compound 1 and 2) or HClO₄ (compound 3) to fall in the range pof pH 2-4.5. A basic environment (pH˜9) was required instead for the synthesis of compound 4. An excess of the pyrophosphate salt was used to help prevent the formation of undesired kinetic products, identified as the dimeric species {[(phen)₂Co]₂(μ-P₂O₇)} in the case of compound 1, or the very stable tris-phen species {[Ni(phen)₃]Cl₂} in the case of compound 2 [{[(phen)₂Co]₂(μ-P₂O₇)}.16H₂O (compound 5) and [Ni(phen)₃]Cl₂.6.5H₂O (compound 6). In each case, the hydrolysis of the pyrophosphate anion has been avoided by reacting the metal salt first with the phen ligand and then with the pyrophosphate salt, following an established strategy in the field.

The structural analysis of compounds 1-4 revealed the complexes typically crystallized as hydrates, with respective formula [Co(phen)₂(H₂P₂O₇)].4H₂O (1.4H₂O), [Ni(phen)₂(H₂P₂O₇)].8H₂O (2.8H₂O), [Cu(phen)(H₂O)(H₂P₂O₇)] (3) and {[Cu(phen)(H₂O)(P₂O₇)] [Na₂(H₂O)₈]}.6H₂O (4.14H₂O). Compounds 1 and 2 crystallize in the triclinic Pi and monoclinic P2₁/c, respectively. The crystal structures are made up of neutral monomeric units of general composition [M(phen)₂(H₂P₂O₇)] (with M=Co(II) in compound 1 and Ni(II) in compound 2) held together by π-π stacking between adjacent phen molecules and hydrogen bonds involving the water molecules of crystallization and the di-hydrogen-pyrophosphate ligand. The metal ion shows a distorted octahedral geometry, being coordinated to two independent cis phen ligands and one di-hydrogen-pyrophosphate group (see FIG. 1(a-b)). The Co—N and Co—O bond distances (Table 2 below) are sufficiently close to those found in the corresponding dimeric species compound 5 (Table 4 below) and in the known Co(II) complexes {[(phen)₂Co]₂(μ-P₂O₇)}.6MeOH (compound 5a) and {[CO₂(μ-P₂O₇)(bpym)₂].12H₂O}ₙ (bpym=2,2′ bipyrimidine), featuring the bridging pyrophosphate tetra-anion, while the Ni—N and Ni—O bond lengths (Table 2) fit well with the reported data for the {[(phen)₂Ni]₂(μ-P₂O₇)}.27H₂O species. A combination of intermolecular phen-phen π-π stacking interaction [interplanar distance in the range 3.3-3.4 Å] and hydrogen bonds between adjacent di-hydrogen-pyrophosphate groups (Table 5) in both structures contribute to define supramolecular channels running along a (compound 1) or c (compound 2) and hosting the molecules of solvent, as seen in FIGS. 1 and 2.

Compound 3 and 4 crystallize in the monoclinic P2₁/n and triclinic Pi space groups, respectively. The crystal structures are made up of neutral (compound 3)/dianionic (compound 4) monomeric units of formula [Cu(phen)(H₂O)(H₂P₂O₇)] and [Cu(phen)(H₂O)(P₂O₇)]⁻², in which the five-coordinated copper(II) ion adopts a classical distorted square-pyramidal geometry (trigonal parameter¹⁵ τ=0.15 for compound 3 and 0.11 for compound 4) with two nitrogen atoms of a single phen ligand and two oxygen atoms of the di-hydrogen (or tetranionic) pyrophosphate group occupying the equatorial positions and a water molecule occupying the apical one. Sodium cations (Table 3 below) and water molecules of crystallization are also present in the crystal lattice of compound 4. The Cu—N and Cu—O bond distances (Table 2 below) are in agreement with published values including the parent dimer {[(phen)Cu(H₂O)]₂(μ-P₂O₇)}.8H₂O}. As in compounds 1 and 2, the phen ligands in compounds 3-4 are involved in significant π-π interactions [interplanar distance of 3.4-3.6 Å] which delineate supramolecular 1D motifs running along the crystallographic a axis, as seen in FIG. 3-4, respectively. An extensive network of hydrogen bonds finally connects these chains in the be plane, ensuring the three-dimensional cohesion (Tables 5-6 below), with the solvent-free 3 network showing a much higher density compared to compounds 1, 2 and 4 (1.93 g cm⁻³ in compound 3 vs. 1.65 g cm⁻³ in both compound 1 and compound 2 and 1.69 g cm⁻³ in compound 4). No substantial differences were noted between compound 4 and the monomeric unit of a bipy analogue {[(bipy)Cu(H₂O)(P₂O₇)Na₂(H₂O)₆].4H₂O} other than the pyrophosphate anion in compound 4 does not coordinate the sodium cations as observed in the bipy structure. In fact, in compound 4, the sodium cations are connected only by bridging water molecules in order to form zig-zag chains, which grow in the b directions, as seen in FIG. 2(b), and contribute to strongly separate the supramolecular monomeric chains along both the b and c axes, as seen in FIG. 3(b).

Table 1 below shows the results obtained from the testing of 1-4 against the drug resistant ovarian cell line A2780/AD. Cisplatin was used as a control and for comparative purposes. The IC₅₀ values (μM) are for compounds 1-4 in A2780/AD cell line. Cisplatin controls and dimeric complexes previously reported are also shown for comparison:

TABLE 1

| | 6 h | 24 h | 72 h | Ref |
|---|---|---|---|---|
| [Co(phen)₂(H₂P₂O₇)] (1) | >1000 | 5 ± 1 | 0.03 ± 0.02 | This work |
| [Ni(phen)₂(H₂P₂O₇)] (2) | >1000 | >1000 | 630 ± 390 | This work |
| [Cu(phen)(H₂O)(H₂P₂O₇)] (3) | 14 ± 6 | 0.7 ± 0.3 | 0.14 ± 0.05 | This work |
| Na₂[Cu(phen)(H₂O)(P₂O₇)] (4) | 15 ± 6 | 0.6 ± 0.6 | 0.08 ± 0.03 | This work |
| Cisplatin | >160 | 200 ± 16 | 13 ± 5 | This work |
| {Co(phen)₂]₂(μ-P₂O₇)} | — | 2 | 1.7 × 10⁻⁴ | 3 |
| {Ni(phen)₂]₂(μ-P₂O₇)} | — | 590 | 304 | 3 |
| {Cu(phen)(H₂O)]₂(μ-P₂O₇)} | — | 0.6 | 1.8 × 10⁻³ | 3 |

While compound 2 revealed little activity, the profiles of compounds 1 and 3/4 show IC₅₀ values reaching the low nanomolar range. However, at earlier time points they exhibit marked differences in cytotoxicities. At 6 hours only the copper(II) species show activity with IC₅₀ values of 14-15 μM, compared to over 1 mM for compound 1 and compound 2. At 24 hours both compound 1 and compound 3 demonstrate low iiM activity, 5 and 0.7 μM, respectively. At 72 hours the cytotoxicity observed for compound 1 and compound 4 becomes statistically similar, between 30 and 80 nM. Note that the two copper(II) species, compounds 3 and 4, have a similar cytotoxicity profile, suggesting that the anionic or neutral nature of the starting material is not critical for activity. The overall differences between the monomeric systems shown here may be attributed to the activation of different cytotoxicity pathways depending on the metal center, as already suggested for the dimeric analogues, with redox chemistry/oxidative stress playing an important role. When comparing the monomers and dimers activities the cytotoxicity of both systems is similar at 24 hours, however the dimers show increased cytotoxicity at 72 hours, suggesting that kinetic control is involved. This also supports the idea that a hydrolysis mechanism is important for the activation of the dimers.

### Example 1

### Synthesis of Compounds 1-4

Solvents and chemicals were of laboratory grade and were used as received. Water was distilled and deionized to 18.6 MX using a Barnstead Diamond Reverse Osmosis machine coupled to a Barnstead Nano Diamond ultrapurification machine. Centrifugation was carried out on a Sorvall RT machine at 4000 rpm for 10 min at room temperature. Infrared spectra were recorded on a Nicolet Magna-IR 850 Series II spectrophotometer as KBr pellets. The relative intensity of reported FT-IR signals are defined as s=strong, br=broad, m=medium and w=weak. Electronic absorption spectra were obtained on a Varian Cary 50 Bio spectrophotometer in 1 mL Quartz cuvettes between 200 nm and 400 nm at room temperature. Thermal analysis was performed on a TA instruments TGA Q500 using 5-10 mg samples placed on platinum pans and ran under a nitrogen atmosphere (40 mL/min). The temperature was ramped from ˜25 to 500° C. at a rate of 5-10° C./min. Analysis was performed using the TA instruments Universal Analysis 2000 software program. Elemental analysis (C, H, N) were performed by QTI d/b/a Intertek, Whitehouse, N.J.

Synthesis of [Co(phen)₂(H₂P₂O₇)].4H₂O (1.4H₂O) and {[(phen)₂Co]₂(μ-P₂O₇)}.16H₂O (5.16H₂O). Cobalt(II) sulphate heptahydrate (0.2811 g, 1 mmol) was dissolved in 25 ml of water, and 1,10-phenanthroline (0.3604 g, 2 mmol) was added, resulting in a dark orange solution upon stifling. A pale peach precipitate immediately formed after the addition of sodium pyrophosphate (0.3989 g, 1.5 mmol) to the cobalt-phen solution (final pH 10.2). Dissolution of this precipitate in methanol and crystallization by vapour diffusion with petroleum ether afforded the literature dinuclear complex {[(phen)₂Co]₂(μ-P₂O₇)}.6MeOH (5.6MeOH=5a). An aqua-solvated analogous species {[(phen)₂Co]₂(μ-P₂O₇)}.16H₂O (5.16H₂O) was obtained by slow evaporation of the aqueous supernatant, yielding light-orange blocks.

Dark-orange block crystals of compound 1 were grown by the slow evaporation at room temperature of a solution obtained by partially re-dissolving the precipitate in water with few drops of concentrate H₂SO₄ (final pH 4.5). The same product can be obtained by using HCl instead of sulphuric acid, and in both cases in the pH range 2-4.5. Analytical data for compound 1 (after drying overnight in vacuo): C₂₄H₂₄CoN₄O₁₀P₂ (MW=649.35): Calcd. C 44.39, H 3.73, N 8.63; Found: C, 44.57; H, 3.09; N, 8.59 (consistent with 3.H₂O). FTIR (KBr): v=3422br, 3065br, 1626w, 1516s, 1425s, 1197s, 1103s, 977w, 916m, 867s, 849s, 727s, 533br cm⁻¹. Analytical data for 5.6H₂O (after drying overnight in vacuo): C₄₈H₄₄CO₂N₈O₁₃P₂ (MW=1120.72): Calcd. C, 51.44; H, 3.96; N, 10.00; Found: C, 51.45; H, 3.52; N, 9.80. FTIR (KBr): v=3404br, 3057br, 1618w, 1515s, 1425s, 1344s, 1217s, 1141s, 1082s, 1013s, 849s, 727s, 637s, 567m cm⁻¹.

Synthesis of [Ni(phen)₂(H₂P₂O₇)].8H₂O (2.8H₂O). Nickel(II) chloride hexahydrate (0.2377 g, 1 mmol) was dissolved in ca. 20 ml of water, and 1,10-phenanthroline was added as a solid (0.3604 g, 2 mmol), under continuous stirring. The resultant light purple colored solution turned grey (final pH 9.9) after the addition of an aqueous solution of anhydrous sodium pyrophosphate (0.5318 g, 2 mmol). The solution (final volume ca. 30 ml) was allowed to stir for 1 h at 50° C. HCl (3M) was then added dropwise, reducing the pH to a value of ca. 2, with a concomitant color change to pink. Light-blue parallelepiped of compound 2 appeared after few days by the slow evaporation of the solvent under ambient conditions. Analytical data for compound 2.3H₂O (after drying overnight in vacuo): C₂₄H₂₄CoN₄O₁₀P₂ (MW=649.11): Calcd. C, 44.41; H, 3.73; N, 8.63; Found: C, 44.73; H, 2.83; N, 8.55. FTIR (KBr): v=3391br, 3068br, 1625w, 1586s, 1517s, 1426s, 1198s, 1106s, 974w, 902m, 870s, 848s, 727s, 532br cm⁻¹.

Synthesis of [Cu(phen)(H₂O)(H₂P₂O₇)] (compound 3) and [Cu(phen)(H₂O)(P₂O₇)] [Na₂(H₂O)₈]}.6H₂O (compound 4.14H₂O). Copper(II) nitrate (0.2326 g, 1 mmol) was dissolved in 20 ml of water, and the ligands 1,10-phenanthroline (0.1802 g, 1 mmol) and pyrophosphate (0.2659 g, 1 mmol of the tetrasodium salt) were added as solids, in stoichiometric quantity, with continuous stirring. The light blue solution of the copper salt and phen turned dark blue after the complete dissolution of the pyrophosphate salt (final pH 9.2). Perchloric acid was added to this solution dropwise, and the pH was lowered to a value of ca. 2. A pale blue precipitate was observed as the pH approached 3, and this was collected by centrifugation and temporarily discarded. Light blue parallelepipeds of compound 3, suitable for X-ray diffractometry study, were obtained by slow evaporation of the pH 2 supernatant, under ambient conditions. The low yield of this reaction can be increased by re-dissolving the low soluble precipitate in water. Blue needles of compound 4.14H₂O were obtained over one week by slow evaporation of the initial solution (pH 9.2). Analytical data for compound 3 (after drying overnight in vacuo): C₁₂H₁₂CuN₂O₈P₂(MW=437.73): Calcd. C, 32.93; H, 2.76; N, 6.40; Found: C, 31.40; H, 2.35; N, 6.27. FTIR (KBr): v=3522m, 3326br, 3240br, 3078w, 1655w, 1520s, 1426s, 1196s, 1148m, 1124s, 984s, 904s, 849s, 743s, 715s, 652s, 543m cm⁻¹. Analytical data for compound 4.4.5H₂O (after drying overnight in vacuo): C₁₂H₁₉CuN₂Na₂O_{12.5}P₂ (MW=562.76): Calcd. C, 25.61; H, 3.40; N, 4.98; Found C, 25.31; H, 3.09; N, 4.98. FTIR (KBr): v=3434br, 1628w, 1520s, 1429s, 1196s, 1146m, 1092s, 1021s, 891w, 724s, 570w cm⁻¹.

Detailed synthesis and characterization. For the synthesis of compounds 1 and 2, M^{II}:phen:P₂O₇⁴⁻ ratios of 1:2:1.5 and 1:2:2 have been used respectively. This led to crystals of compound 2.8H₂O in high yield (ca. 90% based upon NiCl₂), providing the reaction environment was highly acidic (pH close to 2). In the case of the cobalt(II), however, the adopted strategy only reduces the formation of a dimeric pyrophosphate bridged species. Observance of the occurrence of the peach precipitate typically at each tested metal to ligand ratio (1:2:1, 1:2:1.5, 1:2:2, 1:2:3), led to the conclusion that 1:2:1.5 is the optimum compromise between the monomer yield based upon CoSO₄ and the use of excess pyrophosphate salt.

Crystals of compound 1.4H₂O have been obtained with a yield of ca. 45% after partially re-dissolving the precipitate with concentrate H₂SO₄ or HCl, lowering the pH of the solution to ca. 4.5. The yield of this reaction is affected by the occurrence of precipitates at both high and low pH, with the insoluble dimer being the most likely species at high pH (9) and a mixture of various insoluble species in more acidic conditions (pH 2-5). The nature of the initial precipitate has been investigated via re-crystallization from a petroleum ether-methanol vapour diffusion, following the literature procedure,¹ with this process affording the methanol-solvated dimeric species {[(phen)₂Co]₂(μ-P₂O₇)}.6MeOH (compound 5a), as expected. Moreover, the slow evaporation of the supernatant (pH around 9) has provided us with the hydrate analogous {[(phen)₂Co]₂(μ-P₂O₇)}.16H₂O (compound 5.16H₂O), proving the dimer be the most stable species at high pH, even in excess of the pyrophosphate anion. To overcome the problem of the dimer formation we also pursued the synthesis of compound 1 in a constantly acidic environment, with the acid added directly to the metal salt solution, or by using pyrophosphoric acid instead of sodium pyrophosphate, followed by the addition of few drops of 5N NaOH. All these attempts, however, failed in producing a readily identifiable product.

For the synthesis of compounds 3 and 4, CuNO₃.2.5H₂O, phen and Na₄P₂O₇ have been reacted in stoichiometric condition. Crystals of compound 4.14H₂O have been obtained by the slow evaporation of the initial solution (pH around 9) over a period of few days, while a strong acid environment was required for the crystallization of compound 3 (pH lowered to 2 with HClO₄). Similar to what was observed during the synthesis of compound 1, the occurrence of a pale blue precipitate at low pH decreases the yield of this reaction (yield of ca. 15%). However, by strenuously re-dissolving this precipitate in water, the yield can be increased up to ca. 80%. Of note, crystals of both compound 3 and the literature compound {[(phen)Cu(H₂O)]₂(μ-P₂O₇)}.8H₂O}² may be obtained by using various H₂O/MeOH mixtures (1:1, 1:2, 2:1, 3:1) instead of pure water, indicating the presence of methanol hampers the protonation of the pyrophosphate anion, thus favouring the formation of the dimer over the monomer.

The infrared spectra of compounds 1-5 are all very similar, showing a broad band around 3400 cm⁻¹ (presence of lattice water molecules, not observed in the spectrum of compound 3), together with the typical broad and wide bands due to the presence of the pyrophosphate and phen ligands, in the regions 1000-1200 cm⁻¹ and 1400-1550 cm⁻¹ respectively.

During the synthetic attempts to get crystals of complex compound 2, we observed the formation of a pink product even in defect of the phen ligand, when not in presence of an excess of pyrophosphate. Although the pink colour and the IR spectrum indubitably identify this species as a nickel(II) tris-phen monomer, a search in the CCD database revealed that the chloride salt of such catenated has never been reported. For this reason, we also include here the structural analysis of those pink crystals as compound 6 [respective formula [Ni(phen)₃]Cl₂.6.5H₂O].

Crystal structure determination and refinement. X-ray crystallographic data for compounds 1-6 were collected with a Bruker-AXS SMART CCD diffractometer at low temperature (98 K) using graphite monochromated Mo—Ka radiation (λ=0.71073 Å). For data collection and integration, the Bruker SMART and SAINT softwares were employed. Empirical absorption corrections were calculated using SADABS. The structures were solved by Patterson method and subsequently completed by Fourier recycling using the SHELXTL software packages and refined by the full-matrix least-squares refinements based on F² with all observed reflections.

Compounds 1-5. All non-hydrogen atoms were refined anisotropically, except the oxygen atom of the O4w water molecule in the structure of compound 1, which was found disordered over three positions (O4w, O4wa and O4wb, with occupancy factors of 0.5, 0.25 and 0.25, respectively). The hydrogen atoms of the phen ligand were always set in calculated positions and refined using a riding model. The hydrogen atoms on the water molecule of crystallization were usually located on a ΔF map and refined with three restraints for each molecule (two O—H and one H—H distances, 0.96 and 1.43 Å, respectively), with thermal factors fixed to 0.04 Å². No hydrogen atoms have been defined for the water molecules of crystallization in the structure of compound 1. In each case, the water content has been confirmed via TGA analysis, which revealed the presence of 4 (compound 1), 8 (compound 2), 14 (compound 4) and 16 (compound 5) lattice water molecules, in agreement with the compounds crystal structures.

Crystal data for compounds 1-4 are available in CCDC reference numbers 770952-770955. Crystal data (λ=0.71073 Å and T=98(2) K) for compound 5: light orange blocks, C₄₈H₆₄Co₂N₈O₂₃P₂, Mᵣ=1300.87, monoclinic P2ᵢ/n, a=12.852(3), b=21.431(4) and c=21.406(4) Å, β=106.294(4)°, V=5659(1) Å³, Z=4, D_{c}=1.527 g cm³, F(000)=2704, μ(Mo—K_{α})=0.731 mm⁻¹, Refl. collected=48430, Refl. indep. (Rᵢₙₜ)=11558 (0.0559), Refl. obs. [I>2σ(I)]=9222, refinement method=full-matrix least-squares on F², R₁ [I>2σ(I)](all)=0.0438 (0.0590), wR₂[I>2σ(1)] (all)=0.1062 (0.1157), GoF=1.019. CCDC reference number 770956.

Selected bond lengths and angles as well as hydrogen bonds information for each compound are collected in Table 3-7.

Compound 6. All non-hydrogen atoms were refined anisotropically. The hydrogen atoms of the phen ligand were set in calculated positions and refined using a riding model. No hydrogen atoms have been defined for the water molecules of crystallization. The O1w-O9w water molecules reside on the mirror plane and they have a 0.5 occupancy factor. The remaining O10w-O13w molecules reside up or down the plane, in a statistic way; for this reason, their occupancy factors have been set at 0.5 as well. The overall water content has been established on the basis of the structural refinement. The residual maximum density (2.21 eÅ⁻³) in the final Fourier-difference map represents a residual disorder for the O12w water molecule.

Crystal data (λ=0.71073 Å and T=98(2) K) for compound 6: pink parallelepiped, C₃₆H₃₇Cl₂N₆NiO_{6.50}, Mᵣ=787.33, monoclinic C2/m, a=23.416(2), b=21.3012(17) and c=15.4121(12) Å, β=108.133(2)°, V=7305.5(10) Å³, Z=8, D_{c}=1.432 g cm⁻³, F(000)=3272, μ(Mo—K_{α})=0.733 mm⁻¹, Refl. collected=22329, Refl. indep. (Rᵢₙₜ)=8264 (0.0644), Refl. obs. [I>2σ(I)]=4817, refinement method=full-matrix least-squares on F², R₁ [I>2σ(I)](all)=0.0750 (0.1209), wR₂[I>2σ(I)](all)=0.2193 (0.2558), GoF=0.977. CCDC reference number 771373.

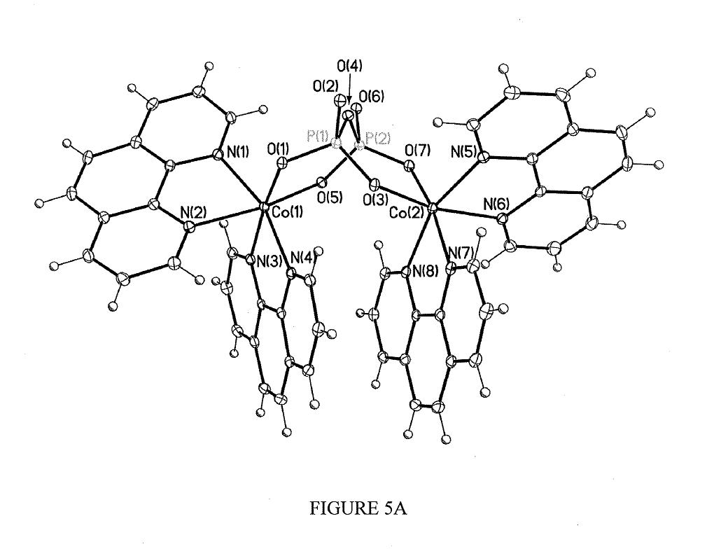

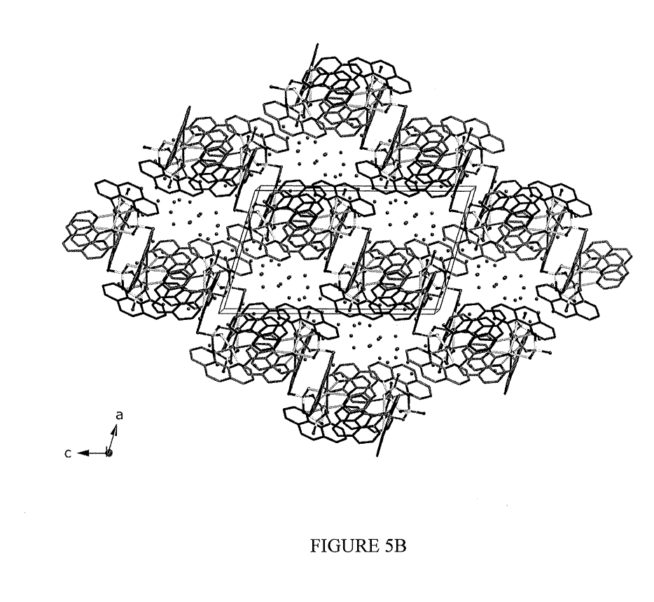

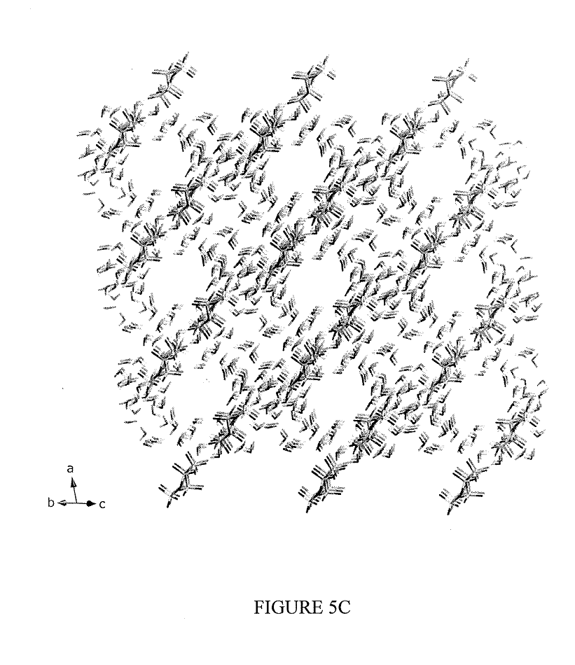

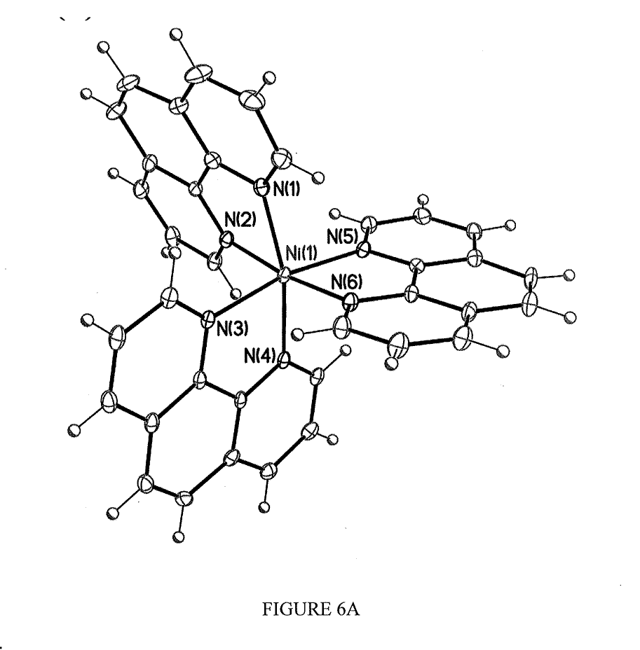

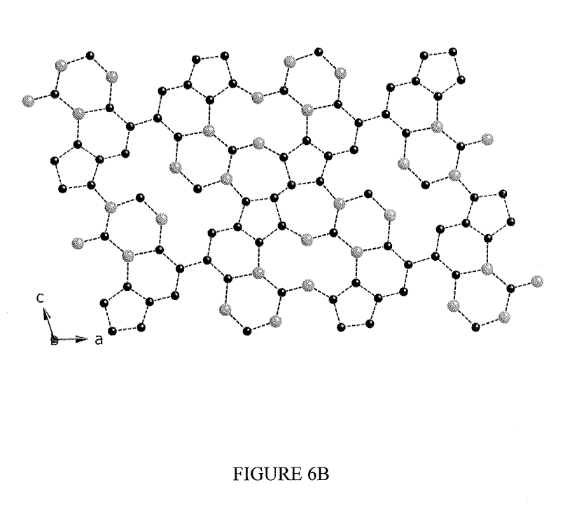

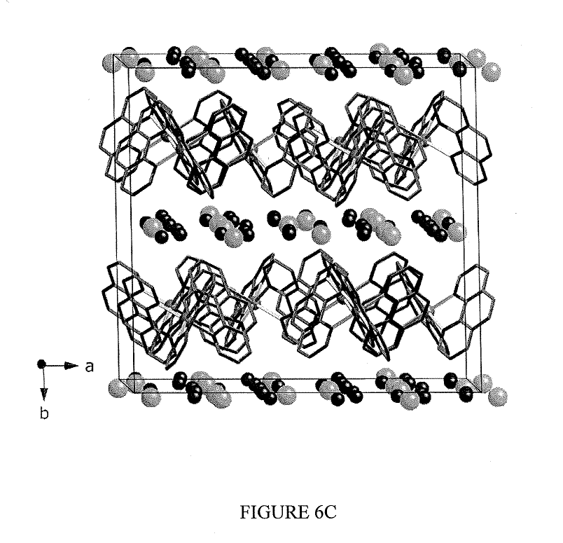

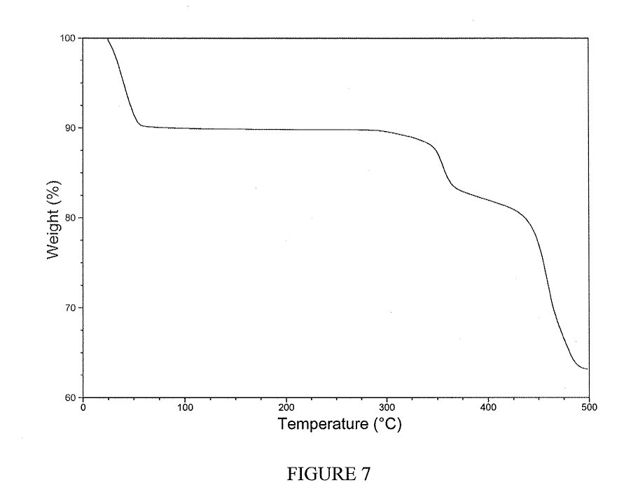

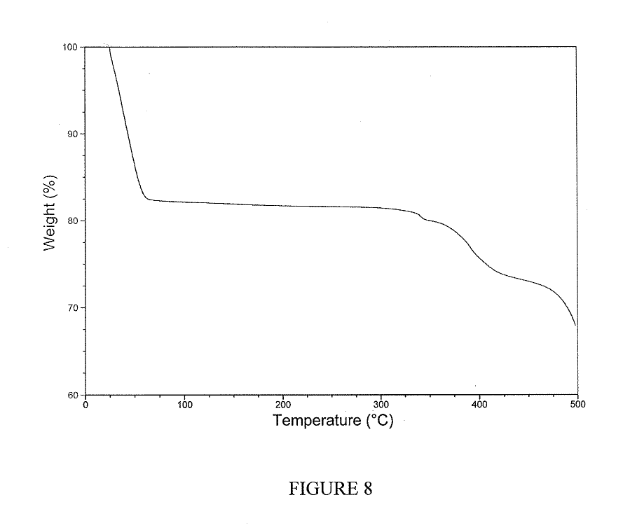

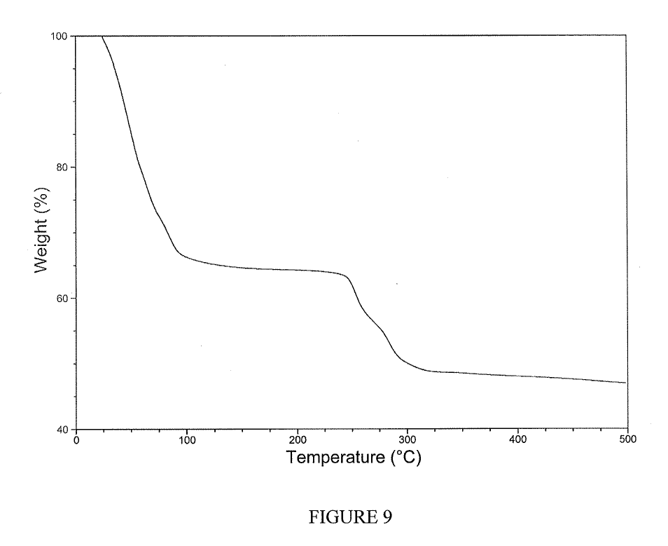

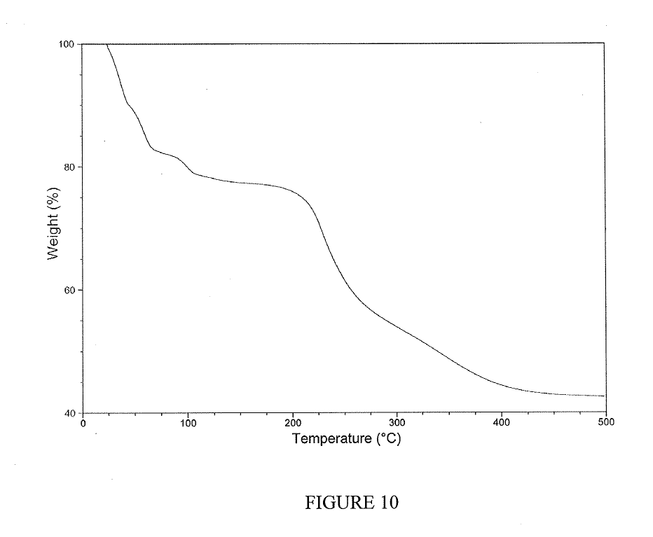

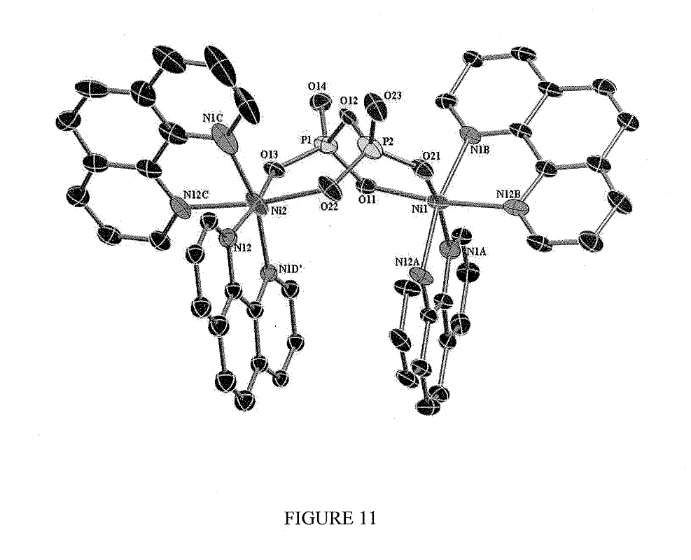

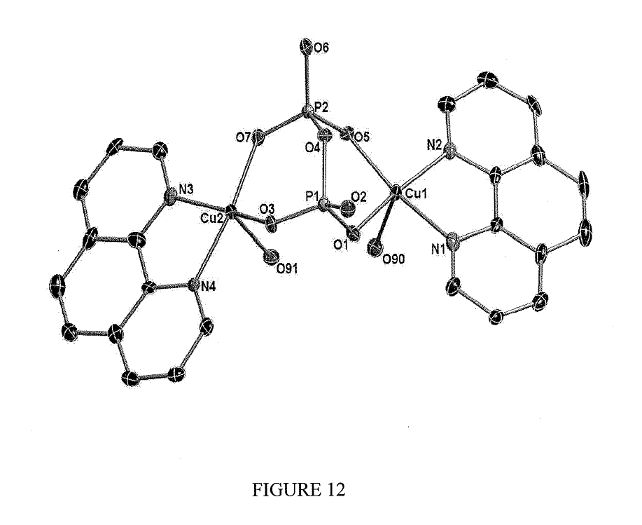

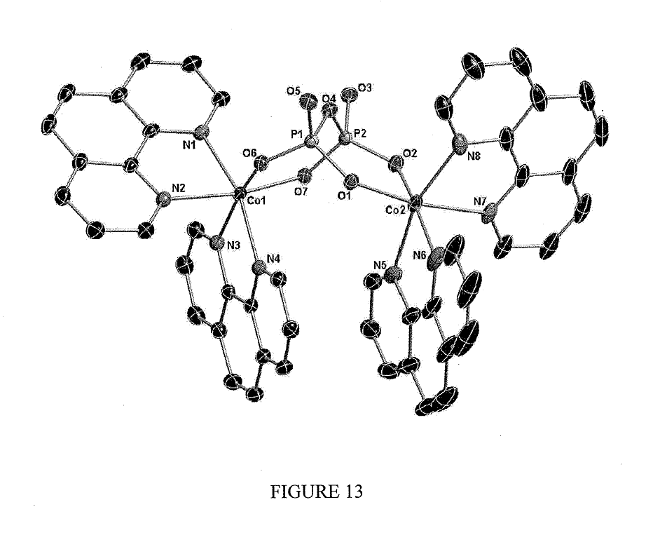

Description of the crystal structure of {[(phen)₂Co]₂(μ-P₂O₇)}.16H₂O (5.16H₂O). Compound 5 crystallizes in the monoclinic P2₁/n space group, whilst the unit cell for the literature methanol solvated compound {[(phen)₂Co]₂(μ-P₂O₇)}.6MeOH (compound 5a) is triclinic PT. Despite the resulting differences within the crystal packing, the dinuclear unit in compound 5 is made up of two crystallographically unique [Co(phen)₂]²⁺ units linked together by the bridging P₂O₇⁴⁻ group, as well as in compound 5a (seen in FIG. 5), with a Co . . . Co separation of 4.645(1) Å [4.857 Å in compound 5a].

The pyrophosphate ligand coordinates to the metal centre in a bis-bidentate manner (“μ” coordination mode) forming two six-membered chelate rings, as already observed in compound 5a and various other dinuclear Mn(II), Ni(II), Cu(II) and Co(III) pyrophosphate complexes. The geometry of the unique cobalt ions [Co(1) and Co(2)] is distorted octahedral with four nitrogen atoms from two phen ligands and two pyrophosphate oxygen atoms, providing each metal center with an N₄O₂ donor set. The Co—N and Co—O bond distances (Table 4) are in agreement with literature values and in particular with those found in compound 5a and in the new monomeric complex [Co(phen)₂(H₂P₂O₇)].4H₂O (compound 1.4H₂O) discussed herein (Table 2). A mean equatorial plane may be defined for each cobalt ion corresponding to the one containing the pyrophosphate oxygen atoms [N(2)-N(3)-O(1)-O(5) for Co(1) and N(6)-N(7)-O(3)-O(7) for Co(2)]. The dihedral angle between these two planes is ca. 87.2°, a value which is significantly lower respect to that observed in compound 5a)(108.2° but closer to the one observed in the manganese(II) complex {[Mn(phen)₂]₂(μ-P₂O₇)}.13H₂O)(88.1°. Providing the crystallization solvent being the only difference between compounds 5 and 5a, this should be ascribed to a packing effect, which in turns may play an important role in determining the magnetic properties of compounds 5 verses 5a. In particular, taking into account parameters as the ion . . . ion separation across the bridging pyrophosphate group and the dihedral angle between the two Co ions' mean planes, a higher ferromagnetic contribute to the magnetic coupling constant J in compounds 5 vs. 5a may be expected.

The crystal packing of compound 5 is dominated by aromatic interaction between the “external” phen ligands and hydrogen bonds involving the pyrophosphate group and the sixteen water molecule of crystallization. The phen ligands are planar and the “internal” ones interact with each other through intramolecular face-to-face 7E-7E stacking with a separation at closet contact of 3.37 Å (3.61 Å in compound 5a) and an overlap efficiency of ca. 33%. The external phen molecules prevalently interact in an edge-to-face manner, defining 1D motifs running along the crystallographic b axis, as seen in FIG. 5(a). Intermolecular face-to-face 7E-7E stacking, with a separation at closet contact of 3.30 Å and an overlap efficiency of ca. 50%, finally contribute to arrange these 1D motifs in the ac plane, resulting in the formation of pseudo-channels filled by the solvent. Indeed, the water molecules of crystallization reside into the channels as well as within them, being involved in the formation of a 3D network of hydrogen bonds together with the pyrophosphate groups (see FIG. 5(b) and Table 7).

TABLE 2
Selected bond lengths (Å) and angles (°) for compounds 1-4.

| | 1•4H₂O | 2•8H₂O | 3 | 4•14H₂O |
|---|---|---|---|---|
| M= | Co(II) | Ni(II) | Cu(II) | Cu(II) |
| Metal | | | | |
| environment | | | | |
| M(1)—O(1w) | — | — | 2.236(2) | 2.218(2) |
| M(1)—O(1) | 2.058(2) | 2.088(2) | 1.944(2) | 1.934(2) |
| M(1)—O(5) | 2.101(2) | 2.058(2) | 1.927(2) | 1.930(2) |
| M(1)—N(1) | 2.143(3) | 2.079(2) | 2.005(2) | 2.032(2) |
| M(1)—N(2) | 2.174(3) | 2.096(2) | 2.015(2) | 2.024(2) |
| M(1)—N(3) | 2.157(3) | 2.110(2) | — | — |
| M(1)—N(4) | 2.109(3) | 2.074(2) | — | — |
| O(1)—M(1)—O(1w) | — | — | 93.82(8) | 92.15(7) |
| O(1)—M(1)—O(5) | 90.8(1) | 90.54(6) | 95.37(8) | 94.41(7) |
| O(1)—M(1)—N(1) | 96.5(1) | 94.16(7) | 90.32(8) | 91.97(8) |
| O(1)—M(1)—N(2) | 96.3(1) | 87.40(6) | 170.39(8) | 172.47(8) |
| O(1)—M(1)—N(3) | 168.4(1) | 173.22(7) | — | — |
| O(1)—M(1)—N(4) | 91.9(1) | 93.66(7) | — | — |
| O(5)—M(1)—O(1w) | — | — | 100.83(8) | 97.46(7) |
| O(5)—M(1)—N(1) | 90.7(1) | 92.76(7) | 161.50(8) | 165.72(8) |
| O(5)—M(1)—N(2) | 166.7(1) | 172.48(7) | 90.43(8) | 91.28(8) |
| O(5)—M(1)—N(3) | 85.7(1) | 89.73(6) | — | — |
| O(5)—M(1)—N(4) | 101.6(1) | 95.06(7) | — | — |
| N(1)—M(1)—O(1w) | — | — | 96.33(8) | 95.05(8) |
| N(1)—M(1)—N(2) | 77.4(1) | 80.20(7) | 81.86(9) | 81.40(8) |
| N(1)—M(1)—N(3) | 94.5(1) | 92.59(7) | — | — |
| N(1)—M(1)—N(4) | 165.0(1) | 168.88(7) | — | — |
| N(2)—M(1)—O(1w) | — | — | 92.61(8) | 92.00(8) |
| N(2)—M(1)—N(3) | 89.4(1) | 93.17(7) | — | — |
| N(2)—M(1)—N(4) | 89.4(1) | 92.29(7) | — | — |
| N(3)—M(1)—N(4) | 78.0(1) | 79.57(7) | — | — |
| Pyrophosphate | | | | |
| environment | | | | |
| P(1)—O(1) | 1.499(2) | 1.513(2) | 1.508(2) | 1.531(2) |
| P(1)—O(2) | 1.491(2) | 1.483(2) | 1.488(2) | 1.497(2) |
| P(1)—O(3) | 1.565(2) | 1.567(2) | 1.561(2) | 1.527(2) |
| P(1)—O(4) | 1.624(2) | 1.617(2) | 1.609(2) | 1.606(2) |
| P(2)—O(4) | 1.603(2) | 1.602(2) | 1.608(2) | 1.621(2) |
| P(2)—O(5) | 1.510(2) | 1.494(2) | 1.505(2) | 1.535(2) |
| P(2)—O(6) | 1.495(2) | 1.498(2) | 1.494(2) | 1.501(2) |
| P(2)—O(7) | 1.569(2) | 1.575(2) | 1.545(2) | 1.520(2) |
| P(1)—O(4)—P(2) | 130.2(1) | 130.0(1) | 128.6(1) | 129.7(1) |
| O(1)—P(1)—P(2)—O(5) | 28.8 | 19.5 | 14.05 | 16.0 |
| O(2)—P(1)—P(2)—O(6) | 35.2 | 134.5 | 18.01 | 113.5 |
| O(3)—P(1)—P(2)—O(7) | 26.0 | 116.9 | 12.09 | 152.1 |

TABLE 3
Sodium environment (bond lengths (Å) and angles (°)) in 4•14H₂O.

| | | | |
|---|---|---|---|
| Na(1)—O(2w) | 2.505(2) | Na(2)—O(6w) | 2.516(2) |
| Na(1)—O(3w) | 2.417(2) | Na(2)—O(7w) | 2.404(2) |
| Na(1)—O(4w) | 2.325(2) | Na(2)—O(8w) | 2.325(2) |
| Na(2)—O(3w) | 2.314(2) | Na(3)—O(7w) | 2.387(2) |
| Na(2)—O(4w) | 2.434(2) | Na(3)—O(8w) | 2.334(2) |
| Na(2)—O(5w) | 2.323(2) | Na(3)—O(9w) | 2.446(2) |
| Na(1) . . . Na(2) | 3.369(2) | Na(2) . . . Na(3) | 3.351(1) |
| O(2w)—Na(1)—O(2wa) | 180 | O(4w)—Na(2)—O(8w) | 78.59(7) |
| O(2w)—Na(1)—O(3w) | 86.66(6) | O(5w)—Na(2)—O(6w) | 97.61(8) |
| O(2w)—Na(1)—O(3wa) | 93.34(6) | O(5w)—Na(2)—O(7w) | 97.59(7) |
| O(2w)—Na(1)—O(4w) | 100.75(6) | O(5w)—Na(2)—O(8w) | 89.88(7) |
| O(2w)—Na(1)—O(4wa) | 79.25(6) | O(6w)—Na(2)—O(7w) | 164.80(8) |
| O(3w)—Na(1)—O(3wa) | 180 | O(6w)—Na(2)—O(8w) | 90.90(7) |
| O(3w)—Na(1)—O(4w) | 89.61(6) | O(7w)—Na(2)—O(8w) | 89.21(7) |
| O(3w)—Na(1)—O(4wa) | 90.39(6) | O(7w)—Na(3)—O(7wb) | 180 |
| O(4w)—Na(1)—O(4wa) | 180 | O(7w)—Na(3)—O(8w) | 89.42(6) |
| O(3w)—Na(2)—O(4w) | 89.48(7) | O(7w)—Na(3)—O(8wb) | 90.58(6) |
| O(3w)—Na(2)—O(5w) | 101.98(7) | O(7w)—Na(3)—O(9w) | 97.10(6) |
| O(3w)—Na(2)—O(6w) | 91.78(7) | O(7w)—Na(3)—O(9wb) | 82.90(6) |
| O(3w)—Na(2)—O(7w) | 84.99(7) | O(8w)—Na(3)—O(8wb) | 180 |
| O(3w)—Na(2)—O(8w) | 167.38(8) | O(8w)—Na(3)—O(9w) | 93.22(6) |
| O(4w)—Na(2)—O(5w) | 168.46(8) | O(8w)—Na(3)—O(9wb) | 86.78(6) |
| O(4w)—Na(2)—O(6w) | 83.25(7) | O(9w)—Na(3)—O(9wb) | 180 |
| O(4w)—Na(2)—O(7w) | 81.87(7) | | |
| Na(1)—O(3w)—Na(2) | 90.77(7) | Na(1)—Na(2)—Na(3) | 109.77(3) |
| Na(1)—O(4w)—Na(2) | 90.10(6) | Na(2)—Na(1)—Na(2a) | 180 |
| Na(2)—O(7w)—Na(3) | 88.76(6) | Na(2)—Na(3)—Na(2b) | 180 |
| Na(2)—O(8w)—Na(3) | 91.98(7) | | |

TABLE 4
Selected bond lengths (Å) and angles (°) for 5•16H₂O.

| Cobalt environment | | | |
|---|---|---|---|
| Co(1)—O(1) | 2.058(2) | Co(2)—O(3) | 2.045(2) |
| Co(1)—O(5) | 2.084(2) | Co(2)—O(7) | 2.079(2) |
| Co(1)—N(1) | 2.151(2) | Co(2)—N(5) | 2.153(2) |
| Co(1)—N(2) | 2.199(2) | Co(2)—N(6) | 2.190(2) |
| Co(1)—N(3) | 2.162(2) | Co(2)—N(7) | 2.173(2) |
| Co(1)—N(4) | 2.145(2) | Co(2)—N(8) | 2.158(2) |
| O(1)—Co(1)—O(5) | 93.16(6) | O(3)—Co(2)—O(7) | 92.37(6) |
| O(1)—Co(1)—N(1) | 94.30(7) | O(3)—Co(2)—N(5) | 89.54(7) |
| O(1)—Co(1)—N(2) | 95.00(7) | O(3)—Co(2)—N(6) | 165.98(7) |
| O(1)—Co(1)—N(3) | 170.50(7) | O(3)—Co(2)—N(7) | 89.85(7) |
| O(1)—Co(1)—N(4) | 93.24(7) | O(3)—Co(2)—N(8) | 105.22(7) |
| O(5)—Co(1)—N(1) | 90.85(7) | O(7)—Co(2)—N(5) | 100.89(7) |
| O(5)—Co(1)—N(2) | 165.19(7) | O(7)—Co(2)—N(6) | 89.76(7) |
| O(5)—Co(1)—N(3) | 90.20(7) | O(7)—Co(2)—N(7) | 167.91(7) |
| O(5)—Co(1)—N(4) | 104.46(7) | O(7)—Co(2)—N(8) | 91.30(7) |
| N(1)—Co(1)—N(2) | 76.25(7) | N(5)—Co(2)—N(6) | 76.45(8) |
| N(1)—Co(1)—N(3) | 94.52(7) | N(5)—Co(2)—N(7) | 91.01(7) |
| N(1)—Co(1)—N(4) | 162.51(7) | N(5)—Co(2)—N(8) | 160.52(7) |
| N(2)—Co(1)—N(3) | 83.71(7) | N(6)—Co(2)—N(7) | 90.96(7) |
| N(2)—Co(1)—N(4) | 87.40(7) | N(6)—Co(2)—N(8) | 88.58(7) |
| N(3)—Co(1)—N(4) | 77.32(7) | N(7)—Co(2)—N(8) | 76.65(7) |
| Pyrophosphate | | | |
| environment | | | |
| P(1)—O(1) | 1.534(2) | P(2)—O(4) | 1.617(2) |
| P(1)—O(2) | 1.501(2) | P(2)—O(5) | 1.519(2) |
| P(1)—O(3) | 1.513(2) | P(2)—O(6) | 1.512(2) |
| P(1)—O(4) | 1.625(2) | P(2)—O(7) | 1.521(2) |
| P(1)—O(4)—P(2) | 122.7(1) | | |

TABLE 5
Hydrogen-bonding interactions in
compound 1•4H₂O, compound 2•8H₂O and compound 3.

| D-H . . . A | D-H/A° | H . . . A/A° | D . . . A/A° | (DHA)/° |
|---|---|---|---|---|
| 1•4H₂O | | | | |
| O(3)—H(3A) . . . O(1w) | 0.82 | 1.83 | 2.647(3) | 178.4 |
| O(7)—H(7A) . . . O(2w)#1 | 0.82 | 1.80 | 2.600(3) | 166.1 |

^{a}Symmetry transformations used to generate equivalent atoms: (#1) −x + 1, −y + 1, −z

| | | | | |
|---|---|---|---|---|
| 2•8H₂O | | | | |
| O(3)—H(3A) . . . O(6)#1 | 0.84 | 1.88 | 2.646(2) | 151.6 |
| O(7)—H(7A) . . . O(2w) | 0.84 | 1.88 | 2.700(2) | 164.4 |
| O(1w)—H(1w) . . . O(6) | 0.94(1) | 1.92(2) | 2.782(2) | 150(2) |
| O(1w)—H(2w) . . . O(7w)#2 | 0.94(1) | 2.05(2) | 2.854(3) | 143(2) |
| O(2w)—H(3w) . . . O(4w)#3 | 0.94(1) | 1.94(1) | 2.857(2) | 162(3) |
| O(2w)—H(4w) . . . O(6w)#1 | 0.95(1) | 1.85(1) | 2.790(3) | 173(2) |
| O(3w)—H(5w) . . . O(4w)#4 | 0.94(1) | 1.85(1) | 2.780(3) | 167(3) |
| O(3w)—H(6w) . . . O(8w)#5 | 0.94(1) | 1.80(1) | 2.731(3) | 170(3) |
| O(4w)—H(7w) . . . O(5w) | 0.94(1) | 1.86(1) | 2.778(3) | 164(2) |
| O(4w)—H(8w) . . . O(2) | 0.94(1) | 1.81(1) | 2.747(2) | 174(3) |
| O(5w)—H(9w) . . . O(1w)#1 | 0.95(1) | 1.79(1) | 2.723(3) | 168(3) |
| O(5w)—H(10w) . . . O(6w)#6 | 0.95(1) | 1.81(1) | 2.742(3) | 168(2) |
| O(6w)—H(11w) . . . O(8w) | 0.93(1) | 1.82(1) | 2.719(3) | 163(3) |
| O(6w)—H(12w) . . . O(3w) | 0.94(1) | 1.79(1) | 2.700(3) | 162(2) |
| O(7w)—H(13w) . . . O(5w)#4 | 0.95(1) | 1.78(1) | 2.728(3) | 176(3) |
| O(7w)—H(14w) . . . O(2w)#1 | 0.94(1) | 2.06(2) | 2.937(3) | 154(3) |
| O(8w)—H(15w) . . . O(1) | 0.94(1) | 1.78(1) | 2.705(2) | 167(3) |
| O(8w)—H(16w) . . . O(1w)#1 | 0.95(1) | 1.87(1) | 2.774(3) | 159(3) |

^{a}Symmetry transformations used to generate equivalent atoms: (#1) −x + 1, −y + 1, −z + 1;
(#2) −x + 1, y − ½, −z + ½; (#3) −x + 1, −y +1, −z + 2; (#4) x, y, z − 1; (#5) x, −y + 3/2, z − ½;
(#6) x, −y + 3/2, z + ½.

| | | | | |
|---|---|---|---|---|
| 3 | | | | |
| O(3)—H(3A) . . . O(1w)#1 | 0.84 | 1.71 | 2.541(2) | 172.6 |
| O(7)—H(7A) . . . O(2)#2 | 0.84 | 1.68 | 2.492(3) | 161.4 |
| O(1w)—H(1w) . . . O(3)#2 | 0.80(3) | 2.15(3) | 2.934(3) | 168(3) |
| O(1w)—H(2w) . . . O(6)#1 | 0.80(3) | 1.97(3) | 2.770(3) | 176(3) |

^{a}Symmetry transformations used to generate equivalent atoms: (#1) x + ½, −y + ½, z + ½;
(#2) x + ½, −y + ½, z − ½

TABLE 6
Hydrogen-bonding interactions in 4•14H₂O.

| D-H . . . A | D-H/A° | H . . . A/A° | D . . . A/A° | (DHA)/° |
|---|---|---|---|---|
| O(1w)—H(1w) . . . O(7)#3 | 0.95(1) | 1.94(1) | 2.878(3) | 174(3) |
| O(1w)—H(2w) . . . O(13w)#3 | 0.94(1) | 1.83(1) | 2.759(3) | 172(3) |
| O(2w)—H(3w) . . . O(3) | 0.94(1) | 1.89(2) | 2.786(3) | 159(3) |
| O(2w)—H(4w) . . . O(1w) | 0.95(1) | 2.28(2) | 3.186(3) | 161(3) |
| O(3w)—H(5w) . . . O(3) | 0.95(1) | 1.77(1) | 2.703(3) | 166(3) |
| O(3w)—H(6w) . . . O(3)#4 | 0.95(1) | 1.90(1) | 2.825(3) | 167(3) |
| O(4w)—H(7w) . . . O(2)#3 | 0.95(1) | 1.91(2) | 2.763(3) | 150(3) |
| O(4w)—H(8w) . . . O(9w)#2 | 0.95(1) | 1.91(1) | 2.829(3) | 163(3) |
| O(5w)—H(9w) . . . O(11w)#5 | 0.95(1) | 1.79(1) | 2.737(3) | 176(3) |
| O(5w)—H(10w) . . . O(6)#6 | 0.95(1) | 1.81(1) | 2.750(3) | 173(4) |
| O(6w)—H(11w) . . . O(2)#3 | 0.95(1) | 2.20(1) | 3.118(3) | 164(3) |
| O(6w)—H(12w) . . . O(1) | 0.94(1) | 1.20(1) | 2.908(3) | 170(3) |
| O(7w)—H(13w) . . . O(2w)#1 | 0.95(1) | 1.93(1) | 2.867(3) | 170(3) |
| O(7w)—H(14w) . . . O(6)#4 | 0.95(1) | 1.88(1) | 2.831(2) | 174(3) |
| O(8w)—H(15w) . . . O(11w) | 0.95(1) | 1.90(1) | 2.838(3) | 172(3) |
| O(8w)—H(16w) . . . O(14w)#6 | 0.95(1) | 1.80(2) | 2.713(3) | 161(3) |
| O(9w)—H(17w) . . . O(6)#6 | 0.95(1) | 1.92(1) | 2.848(3) | 167(3) |
| O(9w)—H(18w) . . . O(5w)#7 | 0.94(1) | 1.88(1) | 2.794(3) | 161(3) |
| O(10w)—H(19w) . . . O(14w)#6 | 0.95(1) | 1.84(1) | 2.759(3) | 163(3) |
| O(10w)—H(20w) . . . O(6w) | 0.95(1) | 1.91(1) | 2.850(3) | 174(3) |
| O(11w)—H(21w) . . . O(2)#3 | 0.94(1) | 1.77(1) | 2.641(3) | 152(3) |
| O(11w)—H(22w) . . . O(15w)#6 | 0.95(1) | 1.99(2) | 2.884(3) | 155(3) |
| O(12w)—H(23w) . . . O(10w) | 0.94(1) | 1.93(2) | 2.837(3) | 162(3) |
| O(12w)—H(24w) . . . O(15w)#6 | 0.95(1) | 1.96(1) | 2.895(3) | 172(3) |
| O(13w)—H(25w) . . . O(12w)#5 | 0.96(1) | 1.92(1) | 2.858(3) | 167(3) |
| O(13w)—H(26w) . . . O(2) | 0.97(1) | 1.80(1) | 2.757(3) | 170(3) |
| O(14w)—H(27w) . . . O(6)#3 | 0.95(1) | 1.81(1) | 2.744(3) | 170(3) |
| O(14w)—H(28w) . . . O(5) | 0.95(1) | 1.78(1) | 2.686(2) | 160(3) |
| O(15w)—H(29w) . . . O(10w)#8 | 0.94(1) | 1.91(1) | 2.839(3) | 169(3) |
| O(15w)—H(30w) . . . O(6)#3 | 0.95(1) | 1.83(1) | 2.770(3) | 174(3) |

^{a} Symmetry transformations used to generate equivalent atoms: (#1) −x + 1, −y + 1, −z + 1; (#2) −x + 1, −y, −z + 1; (#3) x + 1, y, z; (#4) −x, −y + 1, −z + 1; (#5) x − 1, y, z; (#6) x, y − 1, z; (#7) −x, −y, −z + 1; (#8) x + 1, y + 1, z

TABLE 7
Hydrogen-bonding interactions in 5•16H₂O

| D-H . . . A | D-H/A° | H . . . A/A° | D . . . A/A° | (DHA)/° |
|---|---|---|---|---|
| O(1w)—H(1w) . . . O(16w) | 0.95(1) | 1.87(1) | 2.800(3) | 167(3) |
| O(1w)—H(2w) . . . O(6)#1 | 0.95(1) | 1.78(1) | 2.725(3) | 173(3) |
| O(2w)—H(3w) . . . O(6)#1 | 0.96(1) | 1.81(1) | 2.756(3) | 168(3) |
| O(2w)—H(4w) . . . O(8w)#2 | 0.96(1) | 1.82(1) | 2.777(3) | 174(3) |
| O(3w)—H(5w) . . . O(6) | 0.94(1) | 1.85(1) | 2.777(2) | 166(3) |
| O(3w)—H(6w) . . . O(11w) | 0.95(1) | 1.99(1) | 2.921(3) | 168(3) |
| O(4w)—H(7w) . . . O(2w) | 0.96(1) | 1.83(1) | 2.760(3) | 165(3) |
| O(4w)—H(8w) . . . O(7w)#3 | 0.95(1) | 1.81(1) | 2.745(3) | 173(3) |
| O(5w)—H(9w) . . . O(1w) | 0.95(1) | 1.87(1) | 2.804(3) | 170(3) |
| O(5w)—H(10w) . . . O(7)#1 | 0.94(1) | 1.87(1) | 2.798(2) | 167(2) |
| O(6w)—H(11w) . . . O(5w) | 0.95(1) | 2.00(1) | 2.916(3) | 159(3) |
| O(6w)—H(12w) . . . O(7w) | 0.95(1) | 1.95(1) | 2.896(3) | 178(3) |
| O(7w)—H(13w) . . . O(9w)#2 | 0.93(1) | 1.85(1) | 2.745(3) | 159(3) |
| O(7w)—H(14w) . . . O(10w)#4 | 0.94(1) | 1.83(1) | 2.750(3) | 166(2) |
| O(8w)—H(15w) . . . O(11w) | 0.94(1) | 1.97(1) | 2.904(3) | 169(2) |
| O(8w)—H(16w) . . . O(1w)#5 | 0.94(1) | 1.96(1) | 2.838(3) | 156(3) |
| O(9w)—H(17w) . . . O(5w)#5 | 0.94(1) | 1.90(1) | 2.799(3) | 157(2) |
| O(9w)—H(18w) . . . O(13w) | 0.95(1) | 1.78(1) | 2.724(3) | 171(3) |
| O(10w)—H(19w) . . . O(3w)#1 | 0.95(1) | 1.85(1) | 2.797(3) | 175(3) |
| O(10w)—H(20w) . . . O(4w) | 0.95(1) | 1.82(1) | 2.767(3) | 178(3) |
| O(11w)—H(21w) . . . O(9w) | 0.94(1) | 1.89(1) | 2.831(3) | 173(3) |
| O(11w)—H(22w) . . . O(2) | 0.95(1) | 1.80(1) | 2.747(2) | 174(3) |
| O(12w)—H(23w) . . . O(2) | 0.95(1) | 1.78(1) | 2.713(2) | 171(3) |
| O(12w)—H(24w) . . . O(15w)#6 | 0.95(1) | 1.77(1) | 2.713(3) | 171(3) |
| O(13w)—H(25w) . . . O(12w) | 0.95(1) | 1.89(1) | 2.804(3) | 160(2) |
| O(13w)—H(26w) . . . O(1) | 0.95(1) | 1.90(1) | 2.820(2) | 162(3) |
| O(14w)—H(27w) . . . O(12w) | 0.95(1) | 1.87(1) | 2.780(3) | 159(2) |
| O(14w)—H(28w) . . . O(1) | 0.95(1) | 2.03(1) | 2.955(3) | 164(3) |
| O(15w)—H(29w) . . . O(5) | 0.95(1) | 1.83(1) | 2.779(2) | 177(3) |
| O(15w)—H(30w) . . . O(2w)#7 | 0.94(1) | 1.87(1) | 2.806(3) | 172(3) |
| O(16w)—H(31w) . . . O(3w)#1 | 0.95(1) | 2.12(1) | 2.993(3) | 153(2) |
| O(16w)—H(32w) . . . O(14w) | 0.95(1) | 1.86(1) | 2.782(3) | 165(3) |

^{a} Symmetry transformations used to generate equivalent atoms: (#1) x + 1, y, z; (#2) x + ½, −y + ½, z − ½; (#3) −x + 3/2, y − ½, −z + ½; (#4) x − ½, −y + ½, z − ½; (#5) x − ½, −y + ½, z + ½; (#6) x + ½, −y + ½, z + ½; (#7) x − 1, y, z

### Example 2

### Treatment of Cancer Cells with Compounds 1-4

Experimental details for the in-vitro cell testing of compounds [Co(phen)₂(H₂P₂O₇)].4H₂O (1.4H₂O), [Ni(phen)₂(H₂P₂O₇)].8H₂O (2.8H₂O) and [Cu(phen)(H₂O)(H₂P₂O₇)]. For in vitro cell studies, all experiments were performed in a Labconco Purifier I laminar flow hood that had been disinfected with 70% ethanol and irradiated with UV light. The A2780/AD cell line that was used for testing was provided by the Fox Chase Cancer Center, Philadelphia (USA). Fetal bovine serum (FBS) was purchased from Hyclone. Penicillin-streptomycin solution with 10,000 units penicillin and 10 mg/mL streptomycin in 0.9% NaCl was obtained from Sigma. Gibco RPMI 1640 growth media that contained L-glutamine and phenol red was supplied by Invitrogen. Cellgro Cellstripper, a non-enzymatic cell dissociation solution, was obtained from Mediatech. Growth media were filtered with 0.22 μm filter (VWR). Cell Counting Kit 8 (CCK8) was purchased from Dojindo. Cells were incubated and grown in an incubator that was purchased from VWR. Optical densities of the cell cultures were measured at 450 nm with Thermo Multiskan EX that was equipped with Ascent software version 2.6.

Cell lines and culture conditions. The A2780/AD cell line was cultured in BD Falcon 25 cm² surface area, canted neck flaks with vented cap (VWR) containing RPMI media supplemented with 10% FBS and 10% Penicillin-Streptomycin solution by volume. Subcultures were generated by incubating the cells with Cellstripper, and passing cells at a 1:10 culture ratio.

Drug cytotoxicity. Cells used for studies were diluted to make either a solution of 50,000 cells/mL, used for the 72 h studies, or 80,000 cells/mL, used for the 6 and 24 h studies. 100 μL of the cell solution was added to the wells of a 96 well plate, and the cells were allowed to adhere overnight. The media was then removed from the 96 well plates and replaced with either 100 μL (for 6 h studies) or 200 μL (for 24 and 72 h studies) of media containing drug. Drug solutions were prepared by dissolving the drug in media (compounds 2, 4) or in a 25% methanol solution (compounds 1, 3). The solutions were filter sterilized and serially diluted 1:10. The concentration of methanol was kept below 5% for all concentrations studied. Cisplatin was prepared by dissolving in PBS and sonicating for 1 h to create a final concentration of 0.5 mg/mL (1.66 mM). Each drug was tested over 9 concentrations in triplicate with each point in done in triplicate. After the incubation period, the drug containing media was removed and replaced with 100 μL of media containing 10% CCK-8 solution by volume. The plates were incubated until the control reached 0.8-1.2 absorbance at 450 nm (2, 1.5 and 0.5 h for the 6, 24, 72 h runs respectively). The absorbance was measured at 450 nm via a plate reader. A blank was subtracted from all wells, and the results were plotted as a percentage compared to the control vs. concentration of drug. IC₅₀ values were calculated by fitting the data using Prism Graphpad 5 using a nonlinear, dose response curve with a 4 variable fit. The reported ICₛₒ values are an average of at least 3 runs reported with standard deviation.

### Example 3

### Synthesis of Compounds 11-13

Three pyrophosphate bridged complexes, namely {[Ni(phen)₂]₂(μ-P₂O₇)}.27H₂O (compound 11), {[Cu(phen)(H₂O)]₂(μ-P₂O₇)}.8H₂O (compound 12), and {[Co(phen)₂]₂(μ-25 P₂O₇)}.6MeOH (compound 13), (where phen is 1, 1 0′-phenanthroline) also showed highly significant efficacy of cytotoxicity in cancer cell lines such as the adriamycin resistant ovarian cancer cell line A2780/AD, with values as low as 160 pM for the cobalt complex at 72 h. The synthesis and structure of compounds 11-12 is known in the art and was fully set forth in U.S. Provisional App. No. 61/253,815, herein incorporated by reference, and O. F. Ikotun, W. Ouellette, F. Lloret, P. E. Kruger, M. Julve, R. P. Doyle, Eur. J. Inorg. Chem. 17 (2008) 2691-2697, also incorporated by reference herein. Compound 13 may be synthesized from an aqueous suspension of copper(II) nitrate hydrate, 1,10-phenanthroline and sodiumpyrophosphate in a stoichiometric ratio of 2:2:1 to give a blue solution that yielded blue blocks upon standing over several days. The schematic representations of compounds 11-13 may be seen in FIGS. 11 through 13, respectively.

### Example 4

### Treatment of Cancer Cells with Compounds 11-13

Compounds 11, 12, and 13 were tested against the adriamycin-resistant human ovarian cancer cell line (A2780/AD) over a time period of 24 and 72 h (Table 8 below). Cisplatin was used as an external control. Adriamycin resistant ovarian cancer cell line (A2780/AD) were cultured as adherent monolayers in RPMI 1640 (Invitrogen) growth media containing L-glutamine and folic acid supplemented with 10,000 units penicillin, 10 mg/ml streptomycin (Sigma) and 10% (v/v) fetal bovine serum (Sigma). Cells were incubated and grown in a VWR mammalian incubator at 5% CO₂ and 95% humidity. All preparations for cell culture and assays were conducted in a sterile environment under a Labconco Purifier I Laminar flow hood. Cells were cultured in Millipore 250 mL culture bottles with vented lids.

The proliferation of the exponential phase cultures of A2780/AD were assessed by colorimetric assay. WSK-8 (Dojindo) was performed according to manufacturer's instruction. Adherent cell cultures were harvested by stripping of culture flask by a non-enzymatic cell stripper (Mediatech) after a 30 minute incubation period. The cells were then collected. The cell density was adjusted with the addition of RPMI 1640 media to 80,000 cells/mL for exponential growth for the period of drug exposure. To each well, aliquots of 100 μL were inoculated resulting in 8,000 cells per well. After a 24 h incubation time to facilitate adherence, the media was removed and replaced with 200 μl of fresh media containing different concentrations of compounds 11, 12, and 13. The cells were then incubated for 24 and 72 h. Optical densities were measured at 450 nm using a plate reader. The percentage of cell viability was determined relative to untreated control microcultures. The IC₅₀ concentrations were calculated based on a sigmoidal (doseresponse) fit using OriginLabs 8 software with R² values ≧0.70 in all cases (average R² value 0.90). All experimental points were measured in triplicate and each experiment was performed at least three times on separate “batches” of synthesized compound. A2780/AD₂O cells used were fewer than 12 passages for all testing.

Results indicate the nickel derivative, compound 11, exhibits low toxicity marked by the high micromolar inhibitory concentrations observed (see Table 1). In stark contrast however, compounds 12 and 13 were shown to have significant toxicity. Compound 2 was determined to have low nanomolar inhibitory concentrations (see Table 1) while the cobalt analogue 3 proved to be the most toxic with picomolar toxicity, exhibiting an IC₅₀ of ˜162 pM at 72 h.

TABLE 7

| | | IC₅₀ [μM] | |
|---|---|---|---|
| Complex | | 24 h | 72 h |
| {[Ni(phen)₂]₂(μ-P₂O₇)}•27H₂O | 1 | 589.1 ± 31.6 | 304.0 ± 40.9 |
| {[Cu(phen)(H₂O)]₂(μ-P₂O₇)}•8H₂O | 2 | 0.64 ± 0.12 | (1.77 ± 0.15) × |
| | | | 10 − 3 |
| {[Co(phen)₂]₂(μ-P₂O₇)}•6MeOH | 3 | 2.01 ± 0.86 | (1.69 ± 0.54) × |
| | | | 10 − 4 |
| cis-[Pt(NH₃)₂Cl₂] | | 84.0 ± 10.3 | 11.0 ± 1.5 |

Thus, certain compounds of the present invention show effectiveness in treating medical conditions and, more particular, exhibit significant cytotoxicity in exemplary human cancer cell lines.

What is claimed is:

1. A compound comprising a monomeric pyrophosphate bridged metal complex.

2. The compound of claim 1, wherein the metal complex comprises titanium.

3. The compound of claim 1, wherein the metal complex comprises a metal ion selected from the group consisting of nickel, copper, and cobalt.

4. The compound of claim 3, wherein the monomeric pyrophosphate bridged metal complex is selected from the group consisting of [Co(phen)₂(H₂P₂O₇)].4H₂O (1.4H₂O), [Ni(phen)₂(H₂P₂O₇)].8H₂O (2.8H₂O), [Cu(phen)(H₂O)(H₂P₂O₇)], and {[Cu(phen)(H₂O)(P₂O₇)] [Na₂(H₂O)₈]}.6H₂O (4.14H₂O).

5. The compound of claim 4, wherein the monomeric pyrophosphate bridged metal complex is characterized by having cytotoxicity in ovarian cancer cells.

6. The compound of claim 5, wherein the ovarian cancer cells comprise the cell line A2780/AD.

7. A method of treating a medical condition, comprising the steps of administering an effective amount of a pyrophosphate bridged metal complex.

8. The method of claim 7, wherein the metal complex comprises a metal ion selected from the group consisting of nickel, copper, and cobalt.

9. The method of claim 8, wherein the pyrophosphate bridged metal complex comprises a dimeric pyrophosphate bridged metal complex.

10. The method of claim 9, wherein the dimeric pyrophosphate bridged metal complex is a pyrophosphate bridged coordination complex selected from the group consisting of {[Ni(phen)2]2(μ-P2O7)}.27H2O, {[Cu(phen)(H2O)]2(μ-P2O7)}.8H2O, and {[Co(phen)2]2(μ-P2O7)}.6MeOH, wherein phen is 1, 1 0′-phenanthroline.

11. The method of claim 10, the medical condition is ovarian cancer.

12. The method of claim 11, wherein the ovarian cancer is typified by cells of the cell line A2780/AD.

13. The method of claim 8, wherein the pyrophosphate bridged metal complex comprises a monomeric pyrophosphate bridged metal complex.

14. The method of claim 13, wherein the monomeric pyrophosphate bridged metal complex is a selected from the group consisting of [Co(phen)₂(H₂P₂O₇)].4H₂O (1.4H₂O), [Ni(phen)₂(H₂P₂O₇)].8H₂O (2.8H₂O), [Cu(phen)(H₂O)(H₂P₂O₇)], and {[Cu(phen)(H₂O)(P₂O₇)] [Na₂(H₂O)₈]}.6H₂O (4.14H₂O).

15. The method of claim 14, the medical condition is ovarian cancer.

16. The method of claim 15, wherein the ovarian cancer is typified by cells of the cell line A2780/AD.
